(19) United States
(12) Patent Application Publication
(10) Pub. No.: US 2009/0157193 A1
(43) Pub. Date: Jun. 18, 2009

(54) Tendon and Ligament Repair Sheet and Methods of Use

(75) Inventor: William F. McKay, Memphis, TN (US)

Correspondence Address: MEDTRONIC;Attn: Noreen Johnson - IP Legal Department, 2600 Sofamor Danek Drive, MEMPHIS, TN, 38132, US

(73) Assignee: WARSAW ORTHOPEDIC, INC., Warsaw, IN (US)

(21) Appl. No.: 11/958,482

(22) Filed: Dec. 18, 2007

(51) Int. Cl. A61F 2/02; A61B 19/00

(52) U.S. Cl. 623/23.72; 128/898

(57) ABSTRACT

Methods and device for treating or healing an injured tendon or ligament is disclosed. The device, a tendon and ligament repair sheet, has a porous layer and a denser layer, and optionally a therapeutic agent in the porous layer, the denser layer or both. The repair sheet is made from a resorbable or non-resorbable polymer. The repair sheet is securely attached to the injured tendon, ligament, muscle, or bone and has a suture pull out strength of at least 3N. If the injury involve severing of a ligament or tendon, one should place the severed ends in close proximity to each other and securely attach the repair sheet to both sides of the severed tendon or ligament at a distance from the injury so that the repair sheet remains securely attached to the tendon, ligament, muscle, or bone while the tissue is healing.

### FIELD OF THE INVENTION

The present invention relates to a tendon and ligament repair sheet and methods of using it to repair injured tendons and ligaments. More specifically, the tendon and ligament repair sheet is used to repair tendons and ligaments that have been severed or torn.

### BACKGROUND OF THE INVENTION

Tendons are specialized connective soft tissue which connect and attach muscle to bone. Tendons transmit tensile loads from muscle to the attached bone, causing movement of the bone around a joint. Tendons must sustain high tensile forces but be flexible enough to bend around bony surfaces. Ligaments are also specialized connective soft tissue but connect and attach bone to bone. Ligaments provide stability to joints by being flexible enough to allow natural movement of the bones yet also are strong and inextensible to prevent resistance to applied forces. Bundles of collagen fibers are embedded in a connecting matrix, known as ground substance, in tendons and ligaments. These bundles of collagen fibers provide the load carrying elements. In tendons, the collagen fibers are arranged in nearly parallel formation, thus enabling them to withstand high unidirectional loads. In ligaments, the collagen fibers are arranged in a less parallel formation, thereby enabling them to withstand predominant tensile stresses in one direction and smaller stresses in other directions.

Current repair techniques of torn ligaments or tendons involve attaching the severed ends together using sutures or various barbs or anchors. Because the severed ends of the ligament or tendon tend to breakdown prior to the repairing or rejoining of the severed ends, various strips or sleeves have been used in an attempt to help keep the ends attached to each other.

WO 2007/087353, Murray, discloses using a sponge or gel to a torn ligament to help the ligament heal. The sponge or gel is attached to the ligament with a suture and to a bone with a bone screw. This method generates holes in bones and is useful to assisting in the re-attachment of a ligament to a bone. But it is not useful for repairing torn ligaments or tendons when the injury is not adjacent to the bone. It would also not be useful for repairing tendons with tears adjacent to a muscle. Furthermore, the sponges or gel does not provide the dual function of both providing the required structural integrity and optimal delivery of a therapeutic agent.

U.S. Pat. No. 7,179,883, Williams et al., and its related family of patents and applications, disclose methods for producing poly-4-hydroxybutyrate in Escherichia coli. The patent also discloses that the poly-4-hydroxybutyrate can be made into sutures, staples, meshes, patches, slings, pins, barriers, stents, guided tissue repair devices, bulking and filling agents, grafts, and devices for repairing tendons and ligaments using standard techniques for other polymers.

WO 2007/082088, Tarrant et al., discloses the use of a glue to hold the severed ends of a ligament or a tendon together until the ligament or tendon heals. It also discloses a biodegradable, protective sleeve useful in the repair of torn ligaments or tendons. The disclosed sleeve is sutured to the bones on each side of a severed ligament or to the muscle and bone on either side of a severed tendon. This sleeve protects the ligament or tendon from being subjected to the normal forces that a tendon or ligament receives during use. This sleeve acts as a substitute ligament or tendon and allows the torn tissue to heal without receiving the normal stress that occurs during movement. It may be difficult to use a sleeve on each and every ligament or tendon that could possibly be severed. The surrounding tissue may prevent one from placing the sleeve on the severed tendon or ligament or securely attaching the sleeve to the bone or muscle. This sleeve does not provide for optimal delivery of a therapeutic agent. Furthermore, it is well-known that the severed end of a tendon and ligament degrade prior to repairing itself. Glue would not be able to hold the severed ends of the tendon or ligament together during the initial remodeling of the tissue and interfere with healing. Thus, the use of glue is not optimal to keeping the severed ends together during the entire healing process.

U.S. Pat. No. 6,884,428, Binette et al., discloses a foam implant that has a reinforcing knitted mesh material located within the foam. This patent discloses using the implant to help organ regeneration by providing space for cells to grow. Binette et al. discloses adding platelet rich plasma, growth factors and other therapeutic agents to the foam implant. The implant can be placed between the severed ends of a ligament or tendon, or alternatively, wrapped around the tendon. This implant allows the recruitment of too many unwanted cells into the regenerating tissue, thus resulting in scar tissue and/or adhesion formation. It also does not allow for the preferential anatomical release of therapeutic agents to the injury site. Furthermore, it does not allow for the optimization of the device's strength and absorption/release of therapeutic agents by separating these design features into different layers of the device.

Thus, the prior art fails to fulfill a need for a device and method for protecting a severed tendon or ligament during the entire healing process and for keeping the severed ends of the ligament or tendon in close contact with each other during the entire healing process and optimizing the absorption/release of therapeutic agents, thereby reducing or minimizing the amount of scar tissue that will develop.

It is preferable to allow movement of the tendon or ligament shortly after reattachment of the severed ends. This movement helps the patient remain active and allows the new tissue to experience the types of movement and forces which occur naturally. However, one needs to keep the injured ligament or tendon from being stressed too much in order to give the severed ends time to repair themselves.

Fibroblasts may enter into the area between the severed ends of the tendon or ligament. It is hypothesized that scar tissue develops in the repaired tendon or ligament as a result of these fibroblasts that infiltrate into the area between the severed ends. The scar tissue can weaken the tendon or ligament. It may be desirable to prevent such scar formation.

It is also preferable to provide a location distant from the severed ends for attachment of a sheet which can absorb the pressures and stresses of natural movement that occur while the ligament or tendon is healing. If the sheet is attached too close to the site of injury, it may loosen because of the injured tissue is broken down prior to the complete repair of the severed ends.

Tendons and ligaments range in size, and the area in which the present invention may be used can vary (from a finger to hand to shoulder to knee to ankle to foot, etc.). Thus, the tendon and ligament repair sheet must be able to have a range of sizes to conform to the area where the injured ligament or tendon is located. Further, the tendon and ligament repair sheet should be of the appropriate size and shape in order to minimize the drag on surrounding tissue thereby maintaining a smooth area in which the tendon or ligament resides and reducing or minimizing the formation of adhesions.

### BRIEF DESCRIPTION OF THE INVENTION

It is an object of this invention to have a tendon and ligament repair sheet that contains a porous layer and a denser layer which is stronger than the porous layer. The tendon and ligament repair sheet can have openings or holes within the denser layer. The tendon and ligament repair sheet can also have zones of high suture pull-out strength, that are areas which have the strength to withstand high pressure caused by sutures or other securing devices that pull against the repair sheet, thereby not ripping or allowing the suture or other securing device to pull out of the repair sheet. The tendon and ligament repair sheet is attached to a severed ligament or tendon in such a manner that the severed ends are in close proximity to each other so that the severed ends can regenerate and reconnect with each other and such that scar tissue formation is minimized and/or reduced. Because a ligament can tear or become injured close or at to its attachment to the bone, this repair sheet can also be attached at one end to the ligament and at the other end to the bone. Similarly, because a tendon can tear or become injured close or at to its attachment to the bone, or less frequently, close to or at the tendon's attachment to the muscle, this repair sheet can also be attached at one end to the tendon and at the other end to either the bone or the muscle. The repair sheet is attached to the ligament, tendon, bone, and/or muscle at some distance from the severed or torn ends of the tendon or ligament so that the repair sheet remains securely attached to the ligament, tendon, muscle, or bone.

It is another object of this invention that one or more therapeutic agents can be added to the tendon and ligament repair sheet. The one or more therapeutic agents can be on or in the porous layer and/or on or in the denser layer.

An object of this invention is that the tendon and ligament repair sheet is made from resorbable polymers, non-resorbable polymers, or combination of resorbably and non-resorbable polymers.

The tendon and ligament repair sheet can be made from collagen.

The invention involves a method of treating a tendon or ligament having an injury by attaching the tendon or ligament repair sheet to the injured ligament, tendon, muscle, or bone to either sides of the injury so that the tendon or ligament can heal. One or more therapeutic agents can be on or in the repair sheet to assist in the healing of the injury. Because this invention allows local delivery of one or more therapeutic agents, a lower dose of the therapeutic agents can be used compared to the dose of systemically administered therapeutic agents, thus reducing the chance of side effects. One or more therapeutic agents can be on or in the denser layer. One or more therapeutic agents can be on or in the porous layer.

The invention involves a method of preventing the formation of adhesions on an injured tendon or ligament by securing the repair sheet to the injured tendon, ligament, muscle, or bone distal from the injury. One or more therapeutic agents, in or on the repair sheet, assist in the repair of the injured tendon or ligament and/or assist in the prevention of adhesion formation.

### BRIEF DESCRIPTION OF THE FIGURES

FIG. 1A shows one embodiment of the tendon and ligament repair sheet.

FIG. 1B shows another embodiment of the tendon and ligament repair sheet.

FIG. 2 shows a torn tendon.

FIG. 3A shows the tendon and ligament repair sheet in FIG. 1A attached to a torn tendon.

FIG. 3B shows the tendon and ligament repair sheet in FIG. 1B attached to a torn tendon.

### DETAILED DESCRIPTION OF THE INVENTION

The invention described and claimed herein is a sheet for the repair and healing of severed or injured tendons or ligaments. It is referred herein interchangeable as “sheet”, “repair sheet” and “tendon and ligament repair sheet”.

While this invention is described for use in humans, it is anticipated that one may use the invention described herein in animals, including but not limited to mammals, birds, fish, reptiles, and amphibians. It is anticipated that the tendon and ligament repair sheet would be most useful for certain mammals, such as dog, cat, horse, cow, sheep, pig, monkey, ape, chimpanzee, and other mammals for which one may want to repair an injured ligament or tendon. However, this list is not exhaustive, and one could easily use this sheet in any animal. The words “surgeon” and “physician” and “doctor” used herein also mean “veterinarian”.

The term “treating”, “treatment”, “repair”, “repairing”, “heal” or “healing” of a condition described herein refers to executing a protocol, which may include administering one or more drugs to a subject (human or otherwise) and/or performing surgery (minimally invasive or otherwise) on a patient, in an effort to alleviate signs or symptoms of the conditions described herein. These terms also include “preventing” or “prevention” of reoccurrence or occurrence of the conditions described herein, namely the severing of or other type of injury to a ligament or tendon. Reoccurrence may happen when a severed or injured tendon or ligament does not heal properly and is prone to re-injury, pain, and/or tears. In addition, prevention can include inhibiting the formation of scar tissue and/or adhesions that sometimes occur to a tendon or ligament during healing from another type of injury. In addition, “treating” or “treatment” does not require complete alleviation of signs or symptoms, does not require a cure, and specifically includes protocols which have only a marginal effect on the subject.

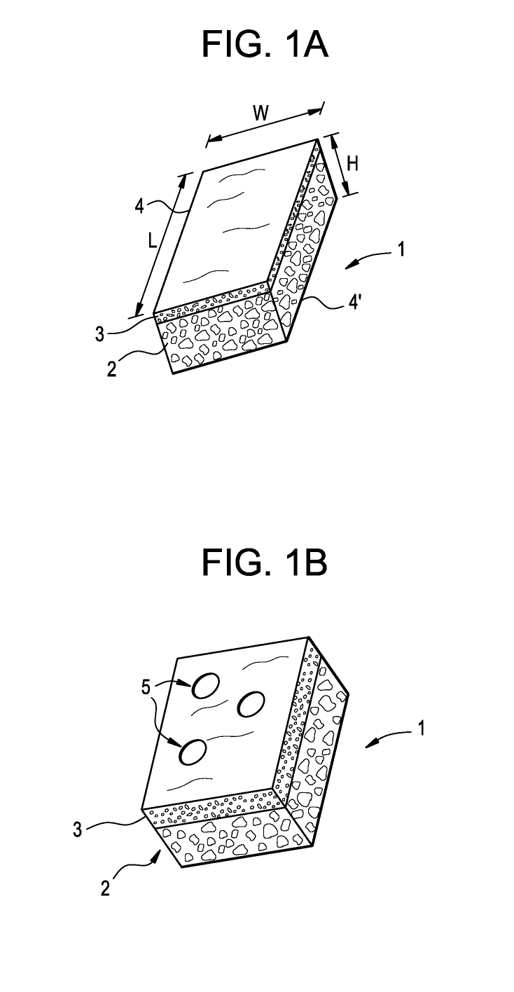

In one embodiment as shown in FIG. 1A, the tendon and ligament repair sheet, 1, has two layers, a porous layer, 2, and a denser layer, 3. The denser layer is stronger than the porous layer. The porous layer, 2, tends to be hydrophilic. Its pores are interconnected to allow for cells to migrate and grow, and to allow for absorption and binding of therapeutic agents. The denser layer has less void space than the porous layer.

The tendon and ligament repair sheet in FIG. 1A has a height, H, width, W, and length, L. The length and width will vary depending of the location within the body where the sheet will be used. It is anticipated that the length is such that one can place the severed ends of the torn ligament or tendon in the middle of the sheet, and the sheet will still extend several centimeters on either side of the tendon, ligament, or bone. The length of the tendon and ligament repair sheet can range from about 1 cm to about 20 cm. The width of the tendon and ligament repair sheet is such that one can wrap from about 25% to more than 100% around the injured tendon or ligament or around the muscle or bone (thus allowing for overlap of the ends of the sheet when wrapped around the injured tendon or ligament). The width can also be such that the tendon and ligament repair sheet wraps around about 60%, about 65%, about 70%, about 75%, about 80%, about 85%, about 90%, about 95%, about 96%, about 97%, about 98%, about 99%, about 100%, about 105%, and about 110% around the torn tendon or ligament. It is understood that when the injury is located adjacent to where a tendon or ligament attaches to a bone or muscle, one may find it difficult to encircle the muscle or bone with the repair sheet. As such, one may place the repair sheet on part of the muscle or bone. The repair sheet may extend from about 5% to about 60%, or about 15% to about 55%, or about 25% to about 50% around a muscle or bone. In certain embodiments, the sheet's width should be sufficient that there is overlap from one edge, 4, over the other edge, 4′, when wrapped around the injured ligament or tendon. However, because it may be difficult to encircle a torn ligament or tendon because of the location of the tendon or ligament, in certain embodiments the sheet does not wrap completely around the tendon or ligament but covers most of the tendon or ligament. Because the size of tendons and ligaments can vary, the width of the sheet can range from about 0.5 cm to about 10 cm. The height, H, of the sheet can range from about 2 mm to about 10 mm. The height should be sufficient that the porous layer has enough porous material to allow for the migration and growth of cells and the binding and release of therapeutic agents. The height should also be sufficient that the denser layer has enough strength to support the sutures, staples or other securing device that attaches the sheet to a tendon, ligament, muscle, or bone. In one embodiment, the denser layer has a suture pull-out strength of at least 3N.

In an alternative embodiment shown in FIG. 1B, one or more openings, 5, exist in the denser layer, 3. The diameter of these openings can vary. The openings, 5, allow the migration of cells through the denser layer, 3, into the porous layer, 2, and to the site of repair of the torn ligament or tendon. The cells may be useful for the repair of the injured tendon or ligament.

In an alternative embodiment, the denser layer may have a suture pull-out strength of less than 3N, equal to 3N, or higher than 3N, yet the denser layer has one, two, three or more zones that have a high suture pull-out strength of at least 3N, at least 4N, at least 5N or at least 6N. The zone should be able to withstand pressure ranging from about 10 to about 1000 N/cm². In this alternative embodiment, the surgeon attaches the tendon and ligament repair sheet to the tendon, ligament, muscle, or bone at these zones of high suture pull-out strength. In the zone(s), the amount of porous layer may be the same as in the other areas of the repair sheet or less than other areas of the repair sheet. While the zone(s) may exist anywhere within the repair sheet, the zone(s) should be located where one attaches the repair sheet to the damaged ligament, tendon, bone, or muscle or where one attaches one side of the repair sheet to the opposite side of the repair sheet. The zone(s) in this embodiment may be highly cross-linked so that it has the high suture pull-out strength. Alternatively, the zone(s) can be made from a polymer distinct from the polymer used to make the denser layer, with the zone's polymer having the ability to withstand pressure ranging from about 10 to about 1000 N/cm². The zone's length and width is such as to provide a sufficient area for the surgeon to comfortably place sutures or staples or other securing devices for attaching the repair sheet to the tendon or ligament. The height can vary, depending on the strength of the material used to form the denser layer. It is anticipated in this embodiment that the height of the zone is equal to or less than the height of the tendon and ligament repair sheet where there is an porous layer and denser layer, that is, the sheet has a uniform height. In some embodiments, the zone(s) acts as a barrier to block the migration of unwanted fibroblasts from entering the porous layer, 2, of the tendon and ligament repair sheet when the repair sheet is attached to an injured ligament or tendon.

The zone of high suture pull-out strength can exist on two, three or four sides of the tendon and ligament repair sheet. When the zones exist on three sides, one can attach the sheet to the injured tendon at the two ends and along part of the third side of the repair sheet. Or, alternatively, the surgeon can attempt to seal the repair sheet around the injured ligament or tendon by attaching the third zone to the repair sheet. When zones exists on all four sides, the surgeon can attach together two adjacent zones, and optionally to areas of the tendon or ligament, to seal the repair sheet around the injured tendon or ligament.

For the various embodiments, the percentage of denser layer to porous layer can range from, but is not limited to, about 100% denser layer and about 0% porous layer, about 90% denser layer and about 10% porous layer, about 80% denser layer and about 20% porous layer, about 70% denser layer and about 30% porous layer, about 60% denser layer and about 40% porous layer, about 50% denser layer and about 50% porous layer, about 40% denser layer and about 60% porous layer, about 30% denser layer and about 70% porous layer, about 20% denser layer and about 80% porous layer, about 10% denser layer and about 90% porous layer, and about 5% denser layer and about 95% porous layer; and any percentage in between these given ranges.

One difference of the present invention over the prior art is that by having two different layers, the porous layer and the denser layer, one layer can be optimized to absorb and release one or more therapeutic agents, while the other layer can be optimized to possess the required strength and fatigue resistance. Prior art inventions lack these desired characteristics in a single device. Also, one could place different therapeutic agents on or in the different layers of the repair sheet.

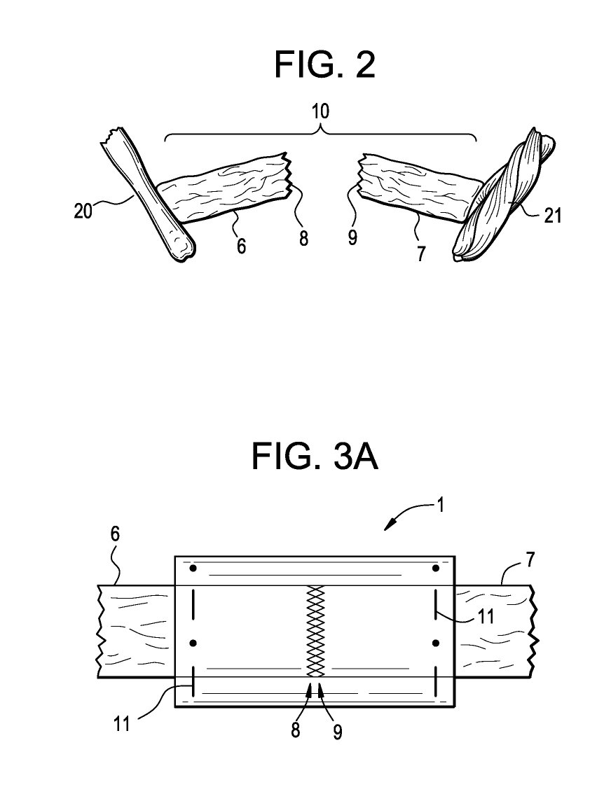

FIG. 2 shows a tendon, 10, severed into two pieces, 6 and 7. The severed ends of the tendon, 8 and 9, are separated from each other. One severed piece, 6, is attached to a bone, 20. The other severed piece, 7, is attached to a muscle, 21.

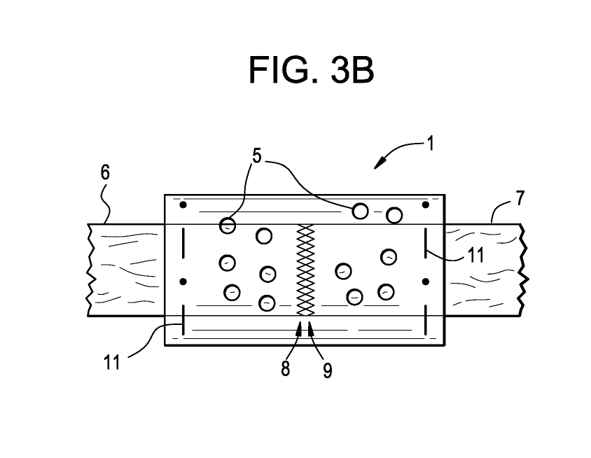

FIG. 3A shows the tendon and ligament repair sheet from FIG. 1A wrapped around a severed tendon, 6 and 7. The porous layer is facing and/or in contact with the tendon (or ligament) while the denser layer is facing the surrounding tissue. The sheet is securely attached at each end to the two pieces of severed tendon at locations sufficiently distant from the severed ends such that the sheet will not become loose or dislodged when the ends of the severed tendon degrade prior to the regeneration and reconnection of the severed tendon. It is anticipated that the repair sheet is secured to the tendon between about 1 cm to about 5 cm from the severed end of the tendon. In one embodiment, the repair sheet is secured to the tendon between about 1 cm and about 4 cm, or about 2 cm from the severed end of the tendon. Sutures, staples, tacks or any other securing device can be used to securely attach the sheet to the tendon. In the pictured embodiment, sutures, 11, have been placed by the surgeon through the denser layer, the porous layer, and the tendon. The denser layer is strong enough so that the sutures, staples, tacks or other securing device does not rip or tear the sheet when the tendon is stressed during movement. Alternatively, sutures can be placed along the length of the repair sheet which may help improve fixation and reduce relative motion between the severed ends of the tendon or ligament. When correctly positioned, the severed ends of the tendon, 8 and 9, are in close proximity to or in physical contact with each other inside the sheet which aids in the repair of the injured tendon.

FIG. 3B shows the tendon and ligament repair sheet, 1, of FIG. 1B attached to a severed tendon. In this embodiment, the tendon and ligament repair sheet, 1, contains openings, 5, in the denser layer. The sheet is wrapped around the torn tendon, 6 and 7. The porous layer is facing and/or is in contact with the tendon (or ligament) while the denser layer is in contact with the surrounding tissue. The sheet is securely attached at each end to the two pieces of severed tendon at locations sufficiently distant from the severed ends such that the sheet will not become loose or dislodged when the ends of the severed tendon degrade prior to the regeneration and reconnection of the severed tendon. Sutures, staples, tacks or any other securing device can be used to securely attach the sheet at the edges to the tendon. In the pictured embodiment, sutures, 11, have been placed by the surgeon through the denser layer, the porous layer, and the tendon. The sutures secure the sheet to the tendon at a site a suitable distance from the severed ends of the tendon. The denser layer is strong enough so that the sutures, staples, tacks or other securing device does not rip or tear the sheet when the tendon is stressed during movement.

Similarly, the repair sheet of FIG. 1A and FIG. 1B can be attached to a severed tendon or ligament and the bone from which the tendon or ligament has torn off. While less frequent, one can also attach the repair sheet to a severed tendon and the muscle from which the tendon has torn off. If the injury is located too close to a bone or muscle such that the surgeon cannot securely attach one end of the repair sheet to the tendon or ligament, the surgeon can attach that end of the repair sheet to the muscle or bone. In order to attach the repair sheet to a bone, the surgeon may need to drill one or more holes into the bone and pass sutures or other attachment devices through the repair sheet and holes in the bone. Alternatively, the surgeon can attach the repair sheet to the bone using an adhesive, staple, or other attachment mechanism. For injuries near or at a muscle, one can attach the repair sheet directly to the muscle.

If the tendon and ligament repair sheet has the high suture pull-out strength zone(s) described above, the surgeon places the sutures, staples or other securing device through the zone(s). In this embodiment, the zone(s) is located on the repair sheet such that the repair sheet is secured, through the zone(s), to the tendon between about 1 cm to about 5 cm from the severed end of the tendon. In one embodiment, the repair sheet is secured to the tendon through the zone between about 1 cm and about 4 cm, or about 2 cm from the severed end of the tendon. Alternatively, the repair sheet can be secured to the severed tendon or ligament through a majority of the length of the repair sheet through a zone that extends the length of the repair sheet. The zone(s) is sufficiently distant from the severed ends such that the repair sheet will not become loose or dislodged when the ends of the severed tendon are degraded prior to the regeneration and reconnection of the severed tendon.

While one embodiment for the tendon and ligament repair sheet is that the porous layer be facing and/or in contact with the injured tendon or ligament while the denser layer is facing the surrounding tissue, another embodiment allows one to attach the repair sheet to the tendon or ligament such that the porous layer is facing the surrounding tissue while the denser layer is facing and/or in contact with the injured ligament or tendon.

It is also an alternative embodiment for the tendon and ligament repair sheet to have a porous layer on both sides of the denser layer. One can either manufacture a repair sheet with the denser layer in between two porous layers or one can stack two repair sheets together such that the two denser layers are adjacent, thus having a porous layer on both sides of the denser layer. One may want to place the same or different therapeutic agents on the two different porous layers.

By securing the tendon and ligament repair sheet to the injured ligament or tendon or to the bone or muscle at one end and the tendon or ligament at the other end, one keeps the severed ends of the ligament or tendon in close proximity to each other so that the ligament or tendon can heal, even while the person is using the severed tendon or ligament. The sheet, when secured to the injured tendon or ligament, prevents the severed ends of the tendon or ligament from pulling away from each other.

One can apply a therapeutic agent to the tendon and ligament repair sheet before the repair sheet is attached to the severed tendon and ligament. Applying a therapeutic agent to the repair sheet allows for localized administration of the therapeutic agent and also allows for one to use a dose that is lower than the dose of same therapeutic agent administered systemically. A benefit of this localized delivery of a therapeutic agent is prevention or reduction of unwanted or adverse side effects from the therapeutic agent. One could apply the therapeutic agent to the porous layer, the denser layer or both, depending on the therapeutic agent, the desired effect, and the manner in which the repair sheet is placed.

The tendon and ligament repair sheet can be made from resorbable or non-resorbable polymers. The polymers can be natural or man-made.

Examples resorbable polymers include, but are not limited to, poly(alpha-hydroxy acids), poly(lactide-co-glycolide) (PLGA), polylactide (PLA), polyglycolide (PG), polyethylene glycol (PEG) conjugates of poly(alpha-hydroxy acids), polyorthoesters, polyaspirins, polyphosphazenes, collagen, elastin, silk, cellulose starch, chitosans, gelatin, alginates, cyclodextrin, polydextrose, dextrans, vinylpyrrolidone, polyvinyl alcohol (PVA), PVA-g-PLGA, polyethyleneglycol-terephtalate and polybuthylene-terephtalate (PEGT-PBT) copolymer (polyactive), methacrylates, poly(N-isopropylacrylamide), polyethylene oxides (as known as polyoxyethylene or PEO), poly-propylene oxide (also known as polyoxypropylene or PPO), poly(aspartic acid) (PAA), PEO-PPO-PEO (Pluronics®, BASF), PEO-PPO-PAA copolymers, PLGA-PEO-PLGA, polyphosphoesters, polyanhydrides, polyester-anhydrides, polyamino acids, polyurethane-esters, polyphosphazines, polycaprolactones, polytrimethylene carbonates, polydioxanones, polyamide-esters, polyketals, polyacetals, glycosaminoglycans, chondroitin sulfate, hyaluronic acid, hyaluronic acid esters, polyethylene-vinyl acetates, silicones, polyurethanes, polypropylene fumarates, polydesaminotyrosine carbonates, polydesaminotyrosine arylates, polydesaminotyrosine ester carbonates, polydesamnotyrosine ester arylates, polyorthocarbonates, polycarbonates, or copolymers or physical blends thereof or combinations thereof.

Non-resorbable polymers can include, but are not limited to, polyethylene, delrin, silicone, polyurethane, copolymers of silicone and polyurethane, polyolefins such as polyisobutylene and polyisoprene, acrylamides such as polyacrylic acid and poly(acrylonitrile-acrylic acid), neoprene, nitrile, acrylates such as polyacrylates, poly(2-hydroxy ethyl methacrylate), methyl methacrylate, 2-hydroxyethyl methacrylate, and copolymers of acrylates with N-vinyl pyrrolidone, N-vinyl lactams, acrylamide, polyurethanes and polyacrylonitrile, glucomannan gel, alkyl celluloses, hydroxyalkyl methyl celluloses, vulcanized rubber and combinations thereof. Examples of polyurethanes include thermoplastic polyurethanes, aliphatic polyurethanes, segmented polyurethanes, hydrophilic polyurethanes, polyether-urethane, polycarbonate-urethane and silicone polyether-urethane. The vulcanized rubber described herein may be produced, for example, by a vulcanization process utilizing a copolymer produced as described, for example, in U.S. Pat. No. 5,245,098 to Summers et al. from 1-hexene and 5-methyl-1,4-hexadiene.

Other suitable non-resorbable material include, but are not limited to, lightly or highly cross-linked biocompatible homopolymers and copolymers of hydrophilic monomers such as 2-hydroxyalkyl acrylates and methacrylates, N-vinyl monomers, and ethylenically unsaturated acids and bases; polycyanoacrylate, polyethylene oxide-polypropylene glycol block copolymers, polygalacturonic acid, polyvinyl pyrrolidone, polyvinyl acetate, polyalkylene glycols, polyethylene oxide, collagen, sulfonated polymers, vinyl ether monomers or polymers, alginate, polyvinyl amines, polyvinyl pyridine, and polyvinyl imidazole. Depending on the amount of crosslinking within the bioresorbable polymers, the degradation time of the polymer can be reduced, thus making the polymer, for the purpose of this invention, appear to be non-resorbable over the time frame of the use of the material for this invention.

In one embodiment, the porous layer, 2, is a porous collagen matrix. The denser layer, 3, is made from highly crosslinked collagen. In alternative embodiments, the porous layer and denser layer can be made using different polymers. In either embodiments, while manufacturing the tendon and ligament repair sheet, one can make a porous layer and denser layer. Then one can laminated together the porous layer and the denser layer using heat, or chemicals, or other suitable laminating techniques. Alternatively, one can form first one layer and then form the other layer directly onto the first layer. For example, one can first form the denser layer and then form the porous layer on top of the denser layer. Alternatively, one can form the porous layer and then form the denser layer on top of the porous layer. In the embodiments that have the edge, the zone of high suture pull-out strength, the zone can be made from the same or different material as the denser layer, although it may be preferable that the zone is made from the same polymer as the denser layer. In those embodiments when openings are present in the denser layer, one can either use a mold to generate the openings during the manufacture of the denser layer or cut the opening into the denser layer after forming the denser layer.

One can optionally add one or more therapeutic agents to the porous layer and/or the denser layer. These therapeutic agents can bind directly to the material of the porous layer or be absorbed within the porous layer, similar to a sponge absorbing water. The therapeutic agents can be bound to the denser layer or absorbed into it. The porous layer and the denser layer can release the therapeutic agents in a sustained release manner or a controlled release manner. A bolus of therapeutic agents can optionally be released shortly after attachment of the sheet to the tendon or ligament with an optional long term release afterward. The tendon and ligament repair sheet can release the therapeutic agents for as long as the repair sheet is present in the body. In some embodiments, the repair sheet will release therapeutic agents from implantation to about two days, or to about 10 days, or to about 20 days, or to about 30 days after implantation. In other embodiments, it will release therapeutic agents for about 2 weeks, about 5 weeks, about 6 weeks, about 10 weeks, about 15 weeks, about 20 weeks or even about 30 weeks after implantation. One can optionally add two or more different therapeutic agents within the porous layer and/or within the denser layer with each therapeutic agent having its own release kinetics. One can add the one or more therapeutic agents to the porous layer and/or to the denser layer shortly before placing the repair sheet inside a patient or before attaching the repair sheet to the severed tendon or ligament. Alternatively, the one or more therapeutic agents can be bound to the porous layer and/or to the denser layer at any point prior to shipping the tendon and ligament repair sheet to the end user.

Examples of therapeutic agents include, but are not limited to, growth factors, cytokine, statins, anti-inflammatory agents, analgesics, antibiotics, mimetics of these therapeutic agents, stem cells or bone marrow cells, any other desirous therapeutic agent, or any combination thereof. As discussed above, because the one or more therapeutic agents are placed in close proximity to the severed tendon ends by virtue of being in the porous layer of the tendon and ligament repair sheet, one can use a lower dosage of the therapeutic agent than if one administered the therapeutic agent systemically. As such, one can avoid the unwanted or adverse side effects of a systemically administered therapeutic agent.

Examples of growth factors can include, but are not limited to, bone morphogenetic protein 12 (BMP-12), BMP-13, BMP-14, members of the BMP family, growth differentiation factor 5 (GDF-5), GDF-6, GDF-7, members of the GDF family, platelet derived growth factor (PDGF), members of the PDGF family, cartilage-derived morphogenetic protein 1 (CDMP-1), CDMP-2, members of the CDMP family, LIM mineralization protein 1 (LMP-1), LMP-3, LMP-3, members of the LMP family, transforming growth factor (TGF) family members, and mimetics of these. The growth factors can be made using recombinant DNA techniques, obtained from animals (mammal, bird, reptile, fish, and amphibian; including but not limited to non-human primates, rat, mouse, hamster, guinea pig, ferret, cow, pig, horse, sheep, dog, cat, chicken, quail, duck, and turkey), or obtained from humans. The BMPs or CDMPs may be obtained from Genetics Institute, Inc., Cambridge, Mass. and may also be prepared by one skilled in the art as described in U.S. Pat. Nos. 5,187,076 to Wozney et al.; 5,366,875 to Wozney et al.; 4,877,864 to Wang et al.; 5,108,922 to Wang et al.; 5,116,738 to Wang et al.; 5,013,649 to Wang et al.; 5,106,748 to Wozney et al.; and PCT Patent Nos. WO93/00432 to Wozney et al.; WO94/26893 to Celeste et al.; and WO94/26892 to Celeste et al., the contents of which are incorporated herein by reference.

Statins inhibit hydroxy-methylglutaryl-coenzyme A reductase (HMG-CoA reductase) and can lower cholesterol in people. Recently, it has been discovered that statins may also help promote growth of specialized connective soft tissue, such as tendons, ligaments, cartilage, and bone. Examples of useful statins for this invention include but are not limited to atorvastatin, cerivastatin, fluvastatin, lovastatin, mevastatin, pitavastatin, rosuvastatin, and simvastatin.

Anti-inflammatory agents can include cytokines, steroids, non-steroidal anti-inflammatories, and agents that inhibit inflammatory cytokines. Of course, these groups can overlap. Examples of agents that inhibit inflammatory cytokines include but are not limited to tumor necrosis factor alpha (TNF-α) inhibitors (onercept, adalimumab, infliximab, etanercept, pegsunercept (PEG sTNF-R1), sTNF-R1, CDP-870, CDP-571, CNI-1493, RDP58, ISIS 104838, 1→3-β-D-glucans, lenercept, PEG-sTNFRII Fc mutein, D2E7, afelimomab and antibodies or antibody fragments that bind to TNF-α or that bind to its receptor), inhibitors of TNF-α production or release (thalidomide, tenidap, and phosphodiesterase inhibitors, such as, but not limited to, pentoxifylline and rolipram), inhibitors of interleukin-1 (IL-1) (anakinra, a recombinant, non-glycosylated form of the human interleukin-1 receptor antagonist (IL-1Ra); Orthokine® (IL-1 Ra obtained from human serum), AMG 108 (a monoclonal antibody that blocks IL-1 activity), and any other antibody or antibody fragment that binds to IL-1 or its receptors), inhibitors of IL-6 (tocilizumab (a humanized anti-IL-6 mAb produced by Chugai) or any other antibody or fragment that binds to IL-6 or its receptor), inhibitors of IL-8 (any antibody or antibody fragment that binds to IL-8 or its receptor), and inhibitors of nuclear factor kappa B (NFκB) (sulindac, clonidine, dexamethasone, flucinolonone, dithiocarbamate, and sulfasalazine).

Cytokines which have anti-inflammatory activity include but are not limited to interleukin-4 (IL-4) IL-10, IL-11, and IL-13.

Examples of steroidal anti-inflammatory agents include but are not limited to hydrocortisone, cortisol, hydroxyltriamcinolone, alpha-methyl dexamethasone, dexamethasone-phosphate, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, difluorosone diacetate, fluocinolone, fluradrenolone acetonide, medrysone, amcinafel, amcinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, and triamcinolone.

Non-limiting examples of non-steroidal anti-inflammatory compounds include acetaminophen, paracetamol, nabumetone, celecoxib, etodolac, nimesulide, apasone, gold, oxicams, such as piroxicam, isoxicam, meloxicam, tenoxicam, sudoxicam, and CP-14,304; the salicylates, such as aspirin, disalcid, benorylate, trilisate, safapryn, solprin, diflunisal, and fendosal; the acetic acid derivatives, such as diclofenac, fenclofenac, indomethacin, sulindac, tolmetin, isoxepac, furofenac, tiopinac, zidometacin, acematacin, fentiazac, zomepirac, clindanac, oxepinac, felbinac, and ketorolac; the fenamates, such as mefenamic, meclofenamic, flufenamic, niflumic, and tolfenamic acids; the propionic acid derivatives, such as ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, and tiaprofenic; and the pyrazoles, such as phenylbutazone, oxyphenbutazone, feprazone, azapropazone, and trimethazone.

Suitable analgesics include, without limitation, non-steroid anti-inflammatory drugs, non-limiting examples of which have been recited above. Analgesics also include other types of compounds, such as, for example, opioids (such as, for example, morphine naloxone, codeine, oxycodone, hydrocodone, diamorphine, and pethidine), local anaesthetics (such as, for example, bipivicain, mupivicain, lidocaine and capsaicin), glutamate receptor antagonists, α-adrenoreceptor agonists (for example, clonidine), adenosine, canabinoids, cholinergic and GABA receptors agonists, and different neuropeptides. A detailed discussion of different analgesics is provided in Sawynok et al., (2003) Pharmacological Reviews, 55:1-20, the content of which is incorporated herein by reference.

One can also place antibiotics on or in the repair sheet to help prevent infection. Examples of antibiotics include but are not limited to amikacin, gentamicin, kanamycin, neomycin, netilmicin, paromomycin, streptomycin, tobramycin and apramycin, streptovaricins, rifamycins, amoxicillin, ampicillin azlocillin, carbenicillin, cloxacillin, dicloxacillin, flucloxacillin, mezlocillin, nafcillin, piperacillin, pivampicillin, ticarcillin, cefacetrile, cefadroxil, cefalexin, cefaloglycin cefalotin, cefapirin cefazolin, cefradine, cefaclor, ceforanide, cefotiam cefprozil, cefuroxime, cefdinir, cefditoren, cefixime cefmenoxime, cefoperazone cefotaxime, cefpiramide, cefpodoxime, ceftazidime, ceftibuten, ceftriaxone, cefepime, cefquinome, sulbactam, tazobactam, clavulanic acid, ampicillin/sulbactam (sultamicillin), co-amoxiclav and combinations thereof.

One can apply stem cells on or to the tendon and ligament repair sheet. Examples of stem cells include pluripotent stem cells, totipotent stem cells, multipotent stem cells, mesenchymal stem cells, bone marrow stem cells, adipose-derived stem cell, and endothelial stem cell. Stem cells may be derived from various tissue sources including, but not limited to, adipose tissue, muscle tissue, peripheral blood, cord blood, blood vessels, skeletal muscle, skin, liver and heart. The tissue may be harvested from autologous, allogeneic or xenogeneic sources; adult or embryonic tissue; a living donor or a cadaver. One can also apply bone marrow cells.

While the stem cells and bone marrow cells may be derived from an autogeneic or from an allogeneic source, the stem cells and bone marrow cells may also be selected from a xenogeneic source. The xenogeneic source is preferably an animal which is closely related to humans, such as a primate, or more preferably, a member of family Hominidae, such as gorilla or chimpanzee. The choice of a non-human source for the stem cells and bone marrow cells may be advantageous because it is possible to produce a large quantity of the stem cells and bone marrow cells of desired type from both embryos and adult animals without legal, ethical, economic, and other concerns accompanying the use of human embryos or adults as the source of the stem cells and bone marrow cells.

Types of therapeutic agents that one may want to attach to the denser layer include, but are not limited to, anti-infiltrating agents, agents to inhibit fibroblast entry into the tendon and ligament repair sheet and severed ends of the tendon or ligament, analgesics, antibiotics, anti-inflammatory agents, and anionic polymers. One can also use the analgesics, antibiotics and anti-inflammatory agents such as those described above.

Anti-infiltrating agents can include, but are not limited to hemostatic agents, anti-adhesion agents, and temporary space occupying barrier materials. Non-limiting examples of hemostatic agents (agents capable of inhibiting or stopping blood flow) include Flowseal® (Fusion Medical Technologies, Mountain View, Calif.), Helistat® (Integra Life Sciences, Plainsboro, N.J.), and Avitene® (Davol, Cranston, R.I.). Non-limiting examples of anti-adhesion agents (agent capable of preventing or inhibiting the formation of post-surgical scar and/or fibrous bands between traumatized tissues and non-traumatized tissue) include Adcon® (Wright Medical Group, Arlington, Tenn.), Oxiplex® (Fziomed, San Luis Obispo, Calif.), Focal Seal® (Genzyme, Cambridge, Mass.), SprayGel™ adhesion barrier system (Confluent Surgical Inc., San Carlos, Calif.), statins, (e.g., lovastatin, simvastatin, pravastatin, fluvastatin, and atorvastatin), anti-VEGF agents (e.g., Avastin® (Genetech, San Francisco, Calif.), Macugen® (Eyetech Pharmaceuticals, Inc., New York, N.Y.), PCK3145 (Procyon BioPharma, Quebec, Calif.), and antibodies to various cytokines or their receptors such as transforming growth factors (such as TGF-α and TGF-β), platelet-derived growth factor, insulin-like growth factors (such as IGF-1 and IGF-2), epidermal growth factor, interleukins, leukocyte derived growth factor, fibroblastic growth factors, vascular endothelial growth factor, heparin-binding epidermal growth factor, and other growth factors involved with wound healing. Of course, one could also use modified versions of a cytokine's receptor as an inhibitor for that cytokine.

Non-limiting examples of temporary space occupying barrier materials include gelatin, PEG, Flogel® (Alliance Pharmaceuticals, San Diego, Calif.), Incert® (Anika Therapeutics, Woburn, Mass.), Hylagel® (Genzyme), Interceed® (Johnson & Johnson, New Brunswick, N.J.), Seprafilm® (Genzyme), Gortex® (W. L. Gore, Newark, Del.), Repel® (Life Medical Sciences, Inc., Edison, N.J.), and Quixil® (Omrix Pharmaceuticals, Inc., New York, N.Y.). Of course, the invention described herein when made from bioresorble material may act as a temporary space occupying barrier.

Anionic polymers may also be useful to inhibit fibrosis, scars, or adhesions. Non-limiting examples of useful anionic polymers include dextran sulfate, pentosan polysulfate, dermatan sulfate, chondroitin sulfate, keratin sulfate, heparin sulfate, heparin and alginate.

The tendon and ligament repair sheet as described above can be manufactured using a variety of techniques that are known in the art to one of ordinary skill in the art field. For example, one can use solvent casting, melt processing, fiber processing/spinning/weaving or other fiber forming extrusion methods, injection and compression molding, lamination, and solvent leaching/solvent cast. One can use an extruder to prepare the invention.

As discussed above, the tendon and ligament repair sheet can be made from natural polymers and synthetic polymers. Of the natural polymers, one may use collagen to make the repair sheet. Collagen is obtained from animals, preferably mammals, such as pigs, cows and sheep. Collagenous tissue in mammals include skin (hide), tendon, intestine, fascia lata, pericardium, and dura mater. One may use the tunica submucosa layer of the small intestine to obtain collagen.

To obtain collagen from the skin or tendon, the animal's skin or tendon is removed from the animal. Extraneous tissue is removed mechanically. Because debris may still be present, the skin or tendon is cut into small pieces, such as 1 cm³. The pieces are frozen at −20° C., then cut into even smaller pieces, for example 1 mm³. Approximately 200 g of minced intestine is suspended in about 1000 ml water. The suspension is known as a slurry and has about pH 6.8. The slurry is heated to around 40° C. Sodium hydroxide, approximately 1.3 ml of 4M NaOH, is added to adjust the pH to pH 8.3. A proteolytic enzyme such as Alcalase® (Novo Industri A/S, Bagsvaerd,. Denmark) is added to the 40° C. slurry. The enzyme hydrolyzes proteins other than collagen. A suitable hydrolysis time is between approximately two to five hours. A suitable quantity of enzyme is about 60 Anson units per kilogram dry substance. The collagen fibers of the skin or tendon are also released during hydrolysis. One can also use detergents such as Triton X-100 (Rohm and Haas, Philadelphia, Pa.) or sodium dodecylsulfate (SDS), enzymes such as dispase, trypsin, or thermolysin, and/or chelating agents such as ethylenediaminetetracetic acid (EDTA) or ethylenebis(oxyethylenitrilo)tetracetic acid (EGTA), to help clean and purify the collagen.

The collagen fibers are collected from the slurry and are washed thoroughly so that both the proteolytic enzyme and hydrolysis products are removed. Any fat remnants can be removed from the collagen, if necessary, by extraction with a solvent. The collagen molecules can be washed with distilled water.

The collagen may optionally be disinfected. One can disinfect the collagen by soaking it in a peracetic acid solution, concentration of about 0.01% to about 0.3% v/v in water, at a neutralized pH of between pH 6 and pH 8. The disinfected collagen may be stored at 4° C. until ready for use.

The collagen fibers are homogenized in a mixer under pressure. The collagen molecules are mixed with liquid to give a slurry with a dryness content of about 1.5% to about 15%. Lactic acid can be added to the slurry to bring the pH to about pH 2.5 to about pH 4.5. To assist release of the individual collagen fibrils, the slurry is homogenized under considerable shearing forces. Then, this the slurry is allowed to mature for about 24 hours. During the maturing process individual collagen fibrils are released. After maturing, the slurry is centrifuged to remove any air bubbles.

To form the denser layer, water is removed to form a dense collagen slurry. This dense collagen slurry is poured into a tray and freeze dried to remove remaining water. Then one, optionally, crosslinks the dense collagen slurry. Crosslinking can be performed using known in the art techniques. Suitable crosslinking agents include, but are not limited to, gluteraldehyde, formaldehyde, 1,4-butanedio diglycidyl ether, hydroxypyridinium, hydroxylysylpyridinium, formalin, and N-(3-dimethylaminopropyl)-N-ethylcarbodiimide hydrochloride (EDC). One can N-hydroxysuccinimide (NHS) when using EDC as a crosslinker. One can also use radiation, heat, or light to crosslink the collagen. One can also use lysyl oxidase or tissue transglutaminase to crosslink the dense layer of collagen. Lysyl oxidase is a metalloprotein which works by crosslinking collagen via oxidative deamination of the epsilon amino groups in lysine.

The denser layer may also be crosslinked by glycation (i.e., the nonenzymatic crosslinking of amine groups of collagen by reducing sugars, such as glucose and ribose) or glycosylation (i.e., the nonenzymatic attachment of glucose to collagen which results in a series of chemical reactions that result in the formation of irreversible crosslinks between adjacent protein molecules). For example, the crosslinks may be pentosidine crosslinks (i.e., crosslinks resulting from the non-enzymatic glycation of lysine and arginine residues). Alternatively, the crosslinks in the collagen can be epsilon(gamma-glutamyl)lysine crosslinks.

If one uses EDC, one would expose the denser layer to 100 mM EDC solution in water overnight. Then one would rinse with water several times to thoroughly remove the EDC and then freeze dry the denser layer again. The crosslinked, dense collagen gives this layer its strength and retards degradation.

If one uses formaldehyde, one would expose the denser layer to formaldehyde gas for several hours. Then one would degas for several hours to thoroughly remove the formaldehyde and freeze dry the denser layer layer.

The porous layer is made from a less dense collagen slurry which can be made by adding water to the slurry, or by not removing as much water as is removed from the slurry used to form the dense collagen layer. To form the porous layer, one can form it directly on the dense collagen layer, or form it separately from the dense collagen layer. To form it on the dense collagen layer, one places the dense collagen layer in a mold, pours the collagen slurry onto the preformed layer and freeze dries it to remove the water. Next one can, optionally, crosslink the porous layer using the techniques described above. One may want to terminate the crosslinking reaction sooner than described above so that the porous layer is not crosslinked as much as the dense layer.

If one wants to form the porous layer separate from the dense layer, one pours the collagen slurry into a mold and freeze dries it. Again, one may optionally crosslink the porous layer with crosslinkers using the techniques described above. Then one needs to fuse or bind the porous layer with the dense layer. One can use heat, pressure, adhesives, chemical linking or other similar techniques to cause this fusion or binding of the two layers. If one uses heat, the temperature and time of heating can depend on the thickness of the layers, the moisture content, and the type of collagen used to make the layers. For example, one can heat the layers at from about 50° C. to about 75° C. for a few minutes to an hour to about 24 hours. Next one cools the tendon and ligament repair sheet using air or water to terminate the binding of the layers. One should be careful that the heating does not denature the collagen fibers and their biological properties. The repair sheet can be stored at any temperature between about 4 to about 25° C. until ready for use.

Prior to use, one opens the package containing the sterile sheet within the sterile operating field. The desired therapeutic agent(s) is reconstituted with sterile water, if necessary, and then is dripped into the porous layer and/or the denser layer, as discussed above.

The repair sheet is wrapped around the circumference of the tendon or ligament tissue bundle as much as possible with the porous side facing the tendon or ligament. The repair sheet is sutured to one part of the torn tendon first. Then as the two ends of the tendon are pulled tight in direct contact with each other, or are slightly overlapped, the other end of the repair sheet is sutured to the other part of the severed tendon or bone. Additional sutures can be placed along the length of the sheet to further attach the sheet to the tendon reducing relative motion of the two torn tendon ends. This technique can be accomplished endoscopically by pre-applying sutures through the repair sheet before introducing the repair sheet into the endoscopic tube leading down to the torn tendon. Then using endoscopic instruments, the sutures can be applied to the torn ends of the tendon and tied off.

The therapeutic agent(s) slowly releases from the porous layer and/or denser layer of the repair sheet, thereby facilitating biological repair of the torn tendon. The repair sheet and sutures slowly degrades over several weeks, transferring tensile loads to the healing tendon. The repair sheet's degradation time can last about six weeks, about eight weeks, about ten weeks, about fifteen weeks, about twenty weeks, and about twenty-five weeks. In some embodiments, the repair sheet may take longer than about twenty-five weeks to degrade.

In an alternative embodiment, the tendon and ligament repair sheet does not degrade over time. Instead, host tissue grows around or through the sheet, or one must operate on the patient (either using minimally invasive technique or open surgery techniques) and remove the repair sheet from the healed tendon or ligament.

While the foregoing discussion teaches the principles of the present invention, with examples provided for the purpose of illustration, it will be appreciated by one skilled in the art from reading this disclosure that variations and changes in form and detail can be made without departing from the scope and nature of this invention.

I, the inventor, claim:

1. A tendon and ligament repair sheet comprising a porous layer and a denser layer, wherein said denser layer is stronger than said porous layer.

2. The repair sheet of claim 1 further comprising at least one therapeutic agent.

3. The repair sheet of claim 2 wherein said therapeutic agent is selected from the group consisting of a growth factor, a cytokine, a statin, an anti-inflammatory agent, a steroid, an analgesic, antibiotic, an anti-infiltrating agent, and a combination thereof.

4. The repair sheet of claim 3 wherein said at least one therapeutic agent is in the porous layer.

5. The repair sheet of claim 3 wherein said at least one therapeutic agent is in or on the denser layer.

6. The repair sheet of claim 1 wherein porous layer and said denser layer contain resorbable polymers, non-resorbable polymers or both resorbable and non-resorble polymers.

7. The repair sheet of claim 6 wherein said porous layer contains collagen and said denser layer contains collagen.

8. The repair sheet of claim 1 wherein said denser layer has a suture pull-out strength of at least 3N.

9. The repair sheet of claim 1 further comprising openings in the denser layer.

10. The repair sheet of claim 1 further comprising at least one zone of high suture pull-out strength.

11. A method of treating a tendon or ligament having an injury comprising
attaching one end of the tendon and ligament repair sheet of claim 1 to one end of said injured tendon or injured ligament; and
attaching the other end of said tendon and ligament repair sheet to a body part selected from the group consisting of the other end of said injured tendon, the other end of said injured ligament, a bone to which said injured ligament or injured tendon is attached, and a muscle to which said injured tendon is attached.

12. The method of claim 11 further comprising adding at least one therapeutic agent to said tendon and ligament repair sheet, wherein said at least one therapeutic agent is selected from the group consisting of a growth factor, a cytokine, a statin, an anti-inflammatory agent, a steroid, an analgesic, antibiotic, an anti-infiltrating agent, a mimetic thereof, and a combination thereof.

13. The method of claim 12 wherein said at least one therapeutic agent is added to the porous layer of said repair sheet.

14. The method of claim 12 wherein said at least one therapeutic agent is added to the denser layer of said repair sheet.

15. A method for treating a severed tendon or ligament comprising
bringing the severed ends of the tendon or ligament in close proximity to each other; and
attaching one end of the tendon and ligament repair sheet of claim 1 to one end of said severed tendon or ligament; and
attaching the other end of the tendon and ligament repair sheet to the other end of severed tendon or ligament or to a bone to which the severed tendon or ligament is connects or to a muscle to which the severed tendon connects at a distance sufficient from said severed end such that said tendon and ligament repair sheet remain securely attached to said severed tendon or ligament during reconnection of the severed ends.

16. The method of claim 15 further comprising adding at least one therapeutic agent to said tendon and ligament repair sheet, wherein said at least one therapeutic agent is selected from the group consisting of a growth factor, a cytokine, a statin, an anti-inflammatory agent, a steroid, an analgesic, antibiotic, an anti-infiltrating agent, a mimetic thereof, and a combination thereof.

17. The method of claim 16 wherein said at least one therapeutic agent is added to the porous layer of said repair sheet.

18. The method of claim 16 wherein said at least one therapeutic agent is added to the denser layer of said repair sheet.

19. The method of claim 15 wherein said attaching step comprises suturing, stapling or tacking said repair sheet to the tendon, ligament, or bone, wherein said denser layer has a suture pull-out strength of at least 3N.

20. The method of claim 15 wherein said repair sheet is wrapped at least about 25% around the severed tendon or ligament.

21. A method of preventing the formation of adhesions on an injured tendon or ligament comprising placing the tendon and ligament repair sheet of claim 1 on the injured tendon or ligament wherein said repair sheet covers the site of injury, and securely attaching said repair sheet to the injured tendon or the injured ligament, or to a bone and to the injured tendon or ligament or to a muscle and to the injured tendon distal to the site of injury.

22. The method of claim 21 further comprising adding at least one therapeutic agent to said tendon and ligament repair sheet, wherein said at least one therapeutic agent is selected from the group consisting of a growth factor, a cytokine, a statin, an anti-inflammatory agent, a steroid, an analgesic, antibiotic, an anti-infiltrating agent, a mimetic thereof, and a combination thereof.

23. The method of claim 22 wherein said at least one therapeutic agent is added to the porous layer of said repair sheet.

24. The method of claim 22 wherein said at least one therapeutic agent is added to the denser layer of said repair sheet.
